(19) United States
(12) Patent Application Publication
(10) Pub. No.: US 2002/0164675 A1
(43) Pub. Date: Nov. 7, 2002

(54) METHODS OF PROPAGATING AND DETECTING INFECTIOUS FORMS OF INTRACELLULAR PATHOGENIC MICROORGANISMS

(76) Inventors: MY LIEN DAO, ST. PETERSBURG, FL (US); BIANCA GARNER, PICKENS, MS (US); KRISTIN S. MORROW, RESIDENCE (XP)

Correspondence Address: CALFEE HALTER & GRISWOLD, LLP, 800 SUPERIOR AVENUE, SUITE 1400, CLEVELAND, OH, 44114, US

(21) Appl. No.: 09/322,547

(22) Filed: May 28, 1999

(51) Int. Cl. C12Q 1/02; C12N 1/00

(52) U.S. Cl. 435/29; 435/243

(57) ABSTRACT

Methods and kits for detecting the presence of infectious forms of intracellular pathogenic protozoa, particularly infectious forms of protozoa in the phylum Microspora and the phylum Apicomplexa, in clinical and environmental samples are provided The methods comprise isolating the protozoa from the test sample; adding an aliquot of the isolated protozoa to the culture medium of a culture of CHO cells, and assaying for the presence of the protozoa within the CHO cells. The kit comprises an antibody that binds to a protein on the infectious form of the protozoa and a sample of CHO cells. Methods of rapidly propagating intracellular pathogenic bacteria and protozoa, particularly protozoa belonging to the phylum Microspora, are also provided. The methods comprise providing a culture of Chinese Hamster Ovary (CHO) cells; adding the protozoa or bacteria to the medium of the cultured cells, and incubating the cells in the presence of the medium for a time sufficient to allow the protozoa or bacteria to infect the cells. Thereafter, the cells are incubated in medium with or without the protozoa or bacteria for a time sufficient for the protozoa or bacterium to replicate within the infected cells. A model system and method for monitoring the effect of candidate pharmacological agents on the infectivity and development of intracellular protozoa and intracellular bacteria are also provided.

### BACKGROUND OF THE INVENTION

Numerous diseases are caused by intracellular pathogenic microorganisms, including intracellular protozoa and intracellular bacteria. In many cases, viable forms of intracellular protozoa and bacteria are present in bodily samples such as, for example, blood, urine, sinonasal secretions, sputum, stools and cerebrospinal fluid, of infected hosts. The contaminated bodily samples may be released into the environment, particularly into the water supply, which can then serve as a source of infection. Viable intracellular pathogenic microorganisms may also be transmitted through transfusion of contaminated blood or blood products derived from infected hosts. Unfortunately, it is difficult to easily and rapidly determine whether such potential sources of infection contain viable and infectious intracellular pathogenic microorganisms. The methods that are currently used to determine whether bodily samples or environmental samples are potential sources of infection can be either time consuming, expensive, or incapable of determining whether the microorganisms that are found in the sample are infectious, i.e., able to enter a host cell and replicate therein. Accordingly, it is desirable to have new methods for detecting the presence of infectious intracellular pathogenic microorganisms in environmental samples and clinical samples.

Infection with intracellular pathogenic protozoa is emerging as a serious worldwide health problem. For example, microsporidia, which is the term used for protozoa belonging to the phylum Microspora, and cryptosporidia, which is the term used for protozoa belonging to the phylum Apicomplexa and genus Cryptosporidium, have both emerged as numerically and clinically important pathogens of the immunocompromised. This large group encompasses AIDS patients, elderly people, young children, patients undergoing immunosuppressive therapy, and malnourished populations of underdeveloped nations. Unfortunately, pharmacological agents for treating infections with microsporidia and cryptosporidia are few. Moreover, the most commonly used agents are not effective against all species of a particular phylum or genus. Thus, proper species identification is an important component of an effective treatment protocol.

At present, the most promising methods for species identification include immunological analysis and genetic analysis. Unfortunately, progress in developing suitable diagnostic tools, i.e. antigens, antibodies, and primers, has been impeded due to the lack of methods for rapidly growing large quantities of these microorganisms. Previously, continuous cultures of different species of intracellular pathogenic protozoa could only be maintained in infected animals, Currently, tissue cultures are used to propagate these intracellular protozoa. However, in the majority of the cell lines that are used for growing microsporidia and cryptosporidia, infections cannot be observed with phase contrast microscopy until 5 days after infection. Moreover, it typically requires 3 to 4 weeks post-infection before mass infectivity occurs. Accordingly, it is desirable to have new methods for propagating intracellular pathogenic protozoa. A method which results in rapid propagation of these microorganisms is especially desirable.

### SUMMARY OF THE INVENTION

The present invention provides a method for detecting the presence of infectious forms of intracellular pathogenic protozoa, particularly infectious forms of protozoa in the phylum Microspora and the phylum Apicomplexa, in clinical and environmental samples. The method comprises isolating the protozoa from the test sample; adding an aliquot of the isolated protozoa to the culture medium of a culture of CHO cells, and assaying for the presence of the protozoa within the CHO cells. In accordance with the present invention, it has been determined that intracellular protozoa, particularly microsporidia and cryptosporidia, rapidly infect and replicate within CHO cells as compared to other cell lines.

The present invention also relates to a kit for detecting the presence of infectious forms of an intracellular protozoa in environmental samples and clinical samples. The kit comprises an antibody that binds to a protein on the infectious form of the protozoa and a sample of CHO cells.

The present invention relates to methods of rapidly propagating intracellular pathogenic bacteria and protozoa, particularly protozoa belonging to the phylum. The method comprises providing a culture of Chinese Hamster Ovary (CHO) cells; adding the protozoa or bacteria to the medium of the cultured cells, and incubating the cells in the presence of the medium for a time sufficient to allow the protozoa or bacteria to infect the cells. Thereafter, the cells are incubated in medium with or without the protozoa or bacteria for a time sufficient for the protozoa or bacterium to replicate within the infected cells.

The present invention also relates to a model system and method for monitoring the effect of candidate pharmacological agents on the infectivity and development of intracellular microorganisms selected from the group consisting of intracellular protozoa and intracellular bacteria.

### BRIEF DESCRIPTION OF THE FIGURES

FIG. 1. Detection of E. intestinalis in CHO cells stained with Gram's stain at 24 hours post inoculation. Uninfected CHO cells stained with (A) complete and (B) partial Gram's stain. Infected CHO cells stained with (C, E) partial and (D, F) complete Gram's stain. Magnification 1000×.

FIG. 2. Detection of E. intestinalis in CHO cells by (A, B) light microscopy and (C, D) scanning electron microscopy at 4 hours post inoculation.

FIG. 3 Detection of E. intestinalis in infected CHO cells by immunofluorescence assay. Uninfected cells at (A) 1, (C) 5 and (E) 10 days. Infected cells at (B) 1, (D) 5, and (F) 10 days post infection. Arrows indicate E. intestinalis spores. Magnification 1000×

FIG. 4. Detection of E. intestinalis in infected CHO cells by phase contrast microscopy at 24 hours post inoculation. (A) Spores with and (B) without filaments. (C) CHO cells at 24 hours post inoculation showing initial infections and (D) mass infections. Magnification 1500×.

FIG. 5 is a graph showing the E. intestinalis spore release pattern in infected CHO cells.

FIG. 6 is a graph is a graph showing production of E. intestinalis spores in infected CHO cells.

FIG. 7. Detection by immunofluorescent assay of C. parvum in infected CHO cells. Two infected CHO cells (A). Infected CHO cell with foci of infection located near CHO cell nucleus (B). Infected CHO cells with possibly two foci of infection within the cytoplasm (C). Normal, control CHO cells, uninfected (D). Magnification 1000×

### DETAILED DESCRIPTION OF THE INVENTION

The present invention provides a method for detecting the presence of viable infectious forms of intracellular pathogenic protozoa in environmental samples, such as water, and clinical samples, such as for example blood or blood products, urine and feces. The method comprises isolating the infectious form of the protozoa from the sample, adding the isolated protozoa to the medium of a culture of CHO cells, and assaying for the presence of the protozoa in the cell. The present method is especially useful for detecting the presence of protozoa belonging to the phylum Microspora and to the genus Cryptosporidium in water supplies.

The phylum Microspora is composed of four genera that are known to cause microsporidosis in humans: Nosema, Enterocytozoon, Pleistophora, and Encephalitozoon. Exemplary species of pathogenic microsporidia include Encephalitozoon cumiculi, Encephalitozoon intestinalis, Encephalitozoon hellem, and Enterocytozoon bieneusi. The infectious stage or form of protozoa in the phylum Microspora consists of spores that range in size measuring between 0.5 to 5 μm in width and 2 to 7 μm in length.

The phylum Apicomplexa includes protozoa in the genus Cryptosporidium. One example of a protozoa in the genus Cryptosporidium which is believed to be responsible for an increasing number of outbreaks of gastrointestinal illness world wide is Cryptosporidium parvum. The infectious form of Cryptosporidium are the sporozoites which excyst from oocysts

Preferably, the infectious form of the protozoa is isolated from the environmental or clinical sample using antibodies which bind to one or more proteins on the outer membrane of the infectious form. Thus, antibodies which bind to spores, oocysts, or sporozoites are used to isolate the infectious form of the protozoa. Optionally, the samples, such as for example water samples or urine samples, and are concentrated by filtration and centrifugation prior to exposure of the sample to the antibody.

Preferably, to increase the specificity of the test the antibody is immunospecific. The term “immunospecific” means the antibodies have substantially greater affinity for one or more outer membrane proteins on the target protozoa than to another protein. Such antibodies may include, but are not limited to, polyclonal, monoclonal, chimeric, single chain, and Fab fragments.

Polyclonal antibodies which bind spores of microsporidia are generated using conventional techniques by administering spores, or homogenates of spores of the microsporidium, to a host animal, preferably a rabbit. Polyclonal antibodies which bind oocysts and sporozoites of cryptosporidia are generated using conventional techniques by administering oocysts, sporozoites or sporozoite membrane from the Cryptosporidium to a host animal. Depending on the host species, various adjuvants may be used to increase immunological response. Conventional protocols are also used to collect blood from the immunized animals and to isolate the serum and or the IgG fraction from the blood.

For preparation of monoclonal antibodies, conventional hybridoma techniques are used. Such antibodies are produced by continuous cell lines in culture. Suitable techniques for preparing monoclonal antibodies include, but are not limited to, the hybridoma technique, the human B-cell hybridoma technique, and the EBV hybridoma technique.

Various immunoassays may be used for screening to identify antibodies having the desired specificity. These include protocols which involve competitive binding or immunoradiometric assays and typically involve the measurement of complex formation between the antigen and the antibody.

For ease of isolation, it is preferred that the antibody be immobilized on immunoabsorbant beads that are used in standard affinity chromatography techniques or on immunomagnetic beads. The efficiencies of two commercially available immunomagnetic bead for isolating Cryptosporidium from environmental samples are examined in an article by Rochelle et al in Appl. Environ. Microbiol (1999) 65(2): 841-845. The infectious forms, which are bound to the antibody, are recovered from the column matrix or the beads using a solution which disrupts the antigen-antibody complex.

A sample or suspension of the isolated spores or oocysts are added to the culture medium of a monolayer of CHO cells which, preferably, is at least 60-70% confluent. Excystation of C. parvum sporozoites from C. parvum oocysts occurs under normal culture conditions. Thus, infection of CHO cells may be initiated by adding either oocysts or sporozoites to the culture medium.

It is preferred that antibiotics and antifungal agents be added to the culture medium prior to addition of the protozoa to the culture medium. Preferably, the protozoa are added to the medium at a ratio of from 1 spore or oocyst/1000 cells to 10 spores or oocysts/1 cell. More preferably, the ratio of protozoa to CHO cells ranges from about 1 to about 10 protozoa per CHO cell.

When the method is used to detect the presence of infectious forms of microsporidia, it is preferred that rabbit polyclonal antibody which binds to and is immunoreactive with a protein on the outer membrane of the microsporidia spore also be added the medium to enhance the rate of infection and replication. When the method is used to detect the presence of infectious forms of cryptosporidia, it is preferred that the recovered oocysts first be pretreated with an excystation fluid. In a preferred embodiment, the excystation fluid contains 0.5% trypsin, 1.5% sodium taurocholate, and 1% acidified Hank's Balanced Salt Solution (pH 7.2) in phosphate-buffered saline (PBS).

The cultures are then incubated for from about 24 to about 48 hours and then assayed to determine whether the isolated protozoa are present within the cells. The preferred assay involves staining reproductive forms of the protozoa and then examining the cells for infected foci. Examples of such stains are 4′,6-diamidino-2-phenylindole (DAPI) fluorescent nucleic acid stain, partial Gram's positive stain, complete Gram's positive stain, Giemsa stain, calcofluor, chromotrope, trichrome blue, and Weber's stain. Such method is also used to quantify the number of infectious microsporidia or cryptosporidia present in the test sample. Alternatively, an immunofluorescent assay is used to detect the presence of the isolated protozoa in the cultured CHO cells. Such assay also permits species identification of the infectious protozoa. Species identification of the infectious protozoa may also be performed using DNA, total RNA, or mRNA which has been extracted from the infected cells, species-specific primers, and standard PCR techniques.

The CHO cell line is an established cell line that is commercially available and easy to culture. In addition, CHO cells have a rapid doubling time and minimal supplemental medium requirements. Accordingly, the present method is advantageously simple and cost effective. Moreover, CHO cells are readily infected with protozoa and support rapid growth of these micro-organisms. For example, infected foci of C. parvum are detectable within 24 hours after inoculation of C. parvum oocysts into the medium of CHO cells and optimal detection is achieved within 48 hours post-inoculation. In contrast, as shown by previous studies, optimal detection of C. parvum infected foci does not occur until 68 hours post-inoculation of C. parvum oocysts into the medium of HCT-8 cells. Similarly, using the present method, infected foci of E. intestinalis are detectable within 4 to 24 hours post-inoculation of E. intestinalis spores into the medium of CHO cells. In contrast, as shown in Table I below, infected foci of E intestinalis and other Encephalitozoon species are not detectable until much later in other cell lines. Accordingly, the present method is also advantageously rapid and time-efficient. Moreover, the present method is also useful for assessing infectivity in test samples that contain low numbers of viable spores or oocysts relative to the number of host cells. Accordingly, the present method is also useful for determining the efficacy of techniques which are designed to kill viable spores and oocysts in environmental samples and clinical samples.

TABLE 1

| Microsporidia | Cell Culture | Detected | Microscopy |
|---|---|---|---|
| E. cuniculi | MRC5 | 3 days | Light stain |
| | HLF | 2-3 weeks | Inverted phase |
| | MDCK | 5 days | Light stain |
| | CDCHME | 2-3 weeks | Inverted phase |
| | FL | 3 days | Light stain |
| | RK13 | 3 days | Light stain |
| | BK | 3 days | Light stain |
| E. hellem | E6 | — | Light stain |
| E. intestinalis | RK13 | 2-3 weeks | Light stain |
| | E6 | 3 weeks | Inverted phase |
| | HEL | 28 days | Light stain |
| | MDM | Never | Light stain |
| Most common cell lines currently used for in vitro growth of microsporidia. The cell lines are MRC5 fibroblasts; human lung fibroblast (HLF); Madin Darby canine kidney (MDCK); human foreskin epithelial (CDCHME); feline lung (FL); rabbit kidney (RK13); bovine kidney (BK); green monkey kidney (E6); human embryonic lung (HEL) and monocyte derived microphages (MDM) (4, 11, 23, 35, 41, 48, 63, 70, 72, 74). Infectivity was never observed in E. hellem infected E6 cells. Spores were only | | | |
| #observed after extrusion into the supernatant. | | | |

The present invention also provides methods for rapidly propagating intracellular pathogenic microorganisms selected from the group consisting of bacteria and protozoa. The methods comprise: providing a culture of CHO cells, adding the bacteria or, preferably, the protozoa, to the medium of said culture, and incubating the cells in the presence of the bacteria or protozoa for a time sufficient to allow the bacteria or protozoa to infect the cells. Thereafter, the cells are incubated in medium with or without the protozoa or bacteria for a time sufficient for the protozoa or bacterium to replicate within the infected cells. Preferably, the method further comprises the step of obtaining the bacteria or protozoa that is produced within the infected cells from the medium.

Preferably, the protozoa or bacteria are added to monolayer cultures of CHO cells that are at least 60-70% confluent. To prevent contamination of the cells with undesirable micro-organisms, it is preferred that antibiotics such as for example, penicillin and streptomycin, and antifungal agents such as, for example, amphotericin B be added to the culture medium prior to addition of the protozoa to the culture medium. Preferably, the protozoa or bacteria are added to the medium at a ratio of from 1 microorganism/1000 CHO cells to 10 micro-organisms/ CHO cell. More preferably, the ratio of protozoa or bacteria to CHO cells ranges from about 1 to about 10 protozoa or bacteria per CHO cell.

The protozoa, particularly microsporidial spores, are isolated from the medium of the cultured cells. The collected pellet is then washed and the spores isolated from cellular debris by centrifugation on a Percoll density gradient.

Good results were obtained when the present method was used to propagate protozoa belonging to the species Encephalitozoon intestinalis. Unexpectedly, massive production of spores was generated within 11 days, yielding results comparable to those achieved when such protozoa are grown for several months in other cell lines. Thus, the present method is especially useful for rapidly propagating protozoa in the phylum Microspora. It has also been determined that the time required for E. intestinalis spores to infect and replicate within CHO cells can be reduced even further when rabbit anti-E. intestinalis antibody is added to the medium along with the spores. Accordingly, it is preferred that rabbit polyclonal antibody to the protozoan spore be added to the medium when the present method is used to propagate protozoa in the phylum Microspora.

The present invention also provides a method of testing candidate pharmacological drugs and determining their efficacy in blocking infection or replication of intracellular pathogenic bacteria and protozoa. As used herein, the term “intracellular” means that the protozoa or bacteria infect and replicate within cells. Examples of bacteria that infect and replicate within cells include Listeria monocytogenes. Examples of protozoa that infect and replicate within cells include, but are not limited to, protozoa belonging to the phylum Microspora and protozoa belonging to the phylum Apicomplexa. The method comprises providing a culture of CHO cells, adding the candidate drug or agent to the medium of the CHO cell culture, inoculating the medium of the CHO cell culture with an inoculum of the bacteria or protozoa, and monitoring the development and/or replication of the bacteria or protozoa within the CHO cell culture. In one embodiment the candidate drug is added to the medium before or concomitantly with the inoculum of the protozoa or bacterium. Such method is used to determine the effect of the candidate on early stages of infection and intracellular replication. In another embodiment, the candidate drug is added to the medium post-inoculation. Preferably, the candidate drug is added from about 4 hour to about 48 hours post-inoculation. Such method is used to determine the effect of the candidate drug on both early and later stages of intracellular replication of the protozoa or bacterium. Preferably, varying concentrations of the candidate drug are added to separate cultures of the CHO cells. Preferably, the method further comprises the step of removing noninternalized protozoa or bacteria. Preferably, non-internalized protozoa or bacteria are removed by replacing the inoculation medium with fresh medium lacking the protozoa or bacteria at from about 24 to about 72 hours post-inoculation.

A number of conventional assay systems/techniques may be used to monitor the effect of the candidate drug on infection or replication. For example, the effect of the candidate drug on infectivity and/or replication can be determined by comparing the number or protozoa and bacteria that are produced in untreated and treated CHO cells. The efficacy of the drug can also be determined by comparing the viability and/or infectivity of micro-organism produced by untreated and treated cells. For microsporidia, the efficacy of the drug can also be determined by observing its effect on spore morphology using electron microscopy and scanning electron microscopy. Such methods are used to test candidate pharmacological agents for intracellular bacteria, such as Listeria monocytogenes, as well as candidate anti-microsporidial agents, and candidate anti-cryptosporidal agents.

The following examples are for purposes of illustration only and are not intended to limit the scope of the claims which are appended hereto.

### EXAMPLE 1

Detection of Microsporidia in CHO Cells by Gram Staining

A. Maintenance of CHO cell cultures and E. intestinalis

CHO cells were maintained in RPMI-1640 (Sigma) supplemented with 200 mM L-Glutamine, 1% sodium pyruvate, 5% fetal calf serum, 100 U penicillin, 10 mg/ml streptomycin, and 25 μg/ml amphotericin B (all from Sigma) in a 5% CO₂ atmosphere at 37° C. in a tissue culture incubator. Cells were grown in 25 and 75-cm² tissue culture flasks (Corning Inc. Corning, N.Y.) and were passaged every 3-4 days by trypsinization with 1 part EDTA and 1 part 0.05% trypsin to disrupt the monolayer. To detect the presence of viable and infectious spores, cells were grown on glass coverslips in 6-well culture chambers in medium containing 2% FCS.

Cultures of E. intestinalis in RK13 cells, obtained from the Department of Soil, Water and. Environmental Science, Univ. Ariz., Tucson, Arizona were maintained in RPMI medium containing 5 % fetal calf serum (FCS) and supplemented as described below. Flasks (75 cm²) were incubated at 37° C. in 5% CO₂. Culture medium was changed weekly and spores were obtained from the collected culture medium by centrifugation for 10 min at 1,000×g. Spores were washed in sterile phosphate buffered saline (PBS) and stored frozen at −70° C., in one ml of medium mixed with one ml of DMSO and 3 ml of fetal calf serum. 100 μl of spore suspension were removed prior to freezing to count the number of spores using a hemocytometer.

B. Inoculation of CHO Cells

Suspensions of 10⁷ CHO cells in 10 ml medium were incubated for 72 h to form a monolayer of over 75% confluency in 100x14 mm culture plates containing three sterile glass cover slips. E. intestinalis spores were thawed from a frozen stock and washed with culture medium to remove DMSO. An inoculum containing 1×10⁵ spores in culture medium was then added into each plate. The cells were then incubated at 37° C. in a 5% CO₂ atmosphere for 1 h, 2 h, 3 h, 4 h, 5 h, 24 h, 48 h, and 10 days. Uninfected plates were incubated in parallel and used as negative controls.

C. Gram staining

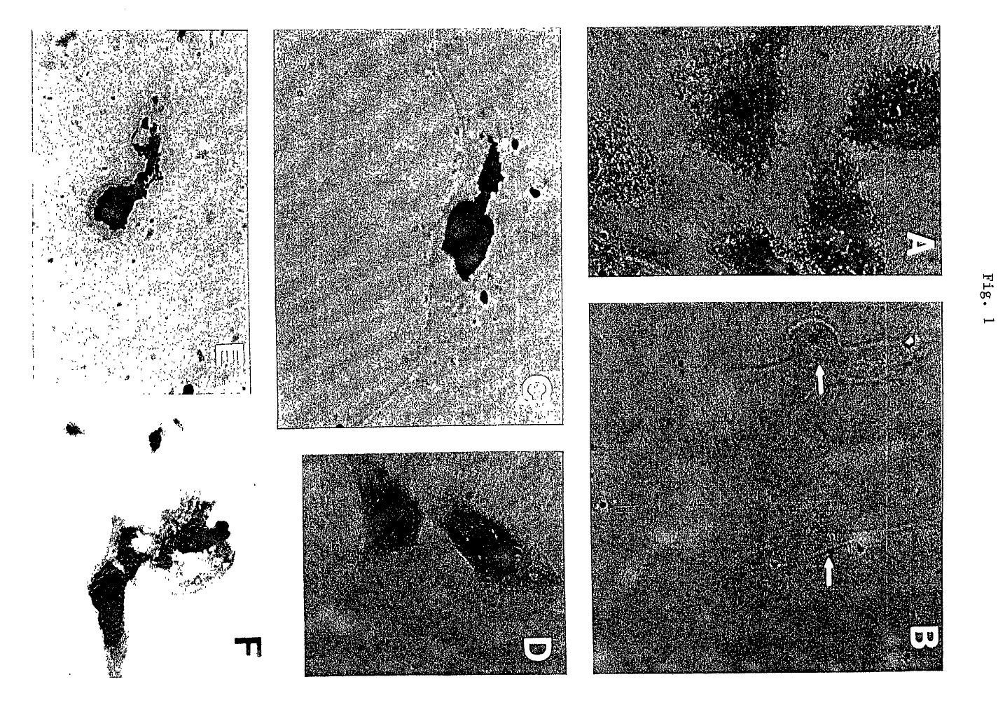

One set of the coverslips inoculated as described above was stained with a partial Gram stain (without counterstain), and one set with complete Gram stain. Cells were examined at 1,000× magnification in a light microscope. With the partial Gram stain, host cells were not visible with the partial Gram stain, while they stained red with the complete Gram stain. Spores stained in purple with both stains. Isolated foci of cells with few spores in small parasitophorous vacuoles were observed 4 h post infection (See FIG. 1). Larger zones of infection were observed at subsequent time points. At 24 h almost all cells were infected with dense vacuoles filled with spores. At 48 h, large zones of infections were present reflecting spreading of infection to adjacent cells with release of exospores. At day 10 post infection, the microscope field was filled with parasitophorous vacuoles containing spores as well as spores being released from these vacuoles. Complete disruption of cell layer was apparent.

### EXAMPLE 2

Detection of Microsporidia in CHO Cells Via Electron Microscopy

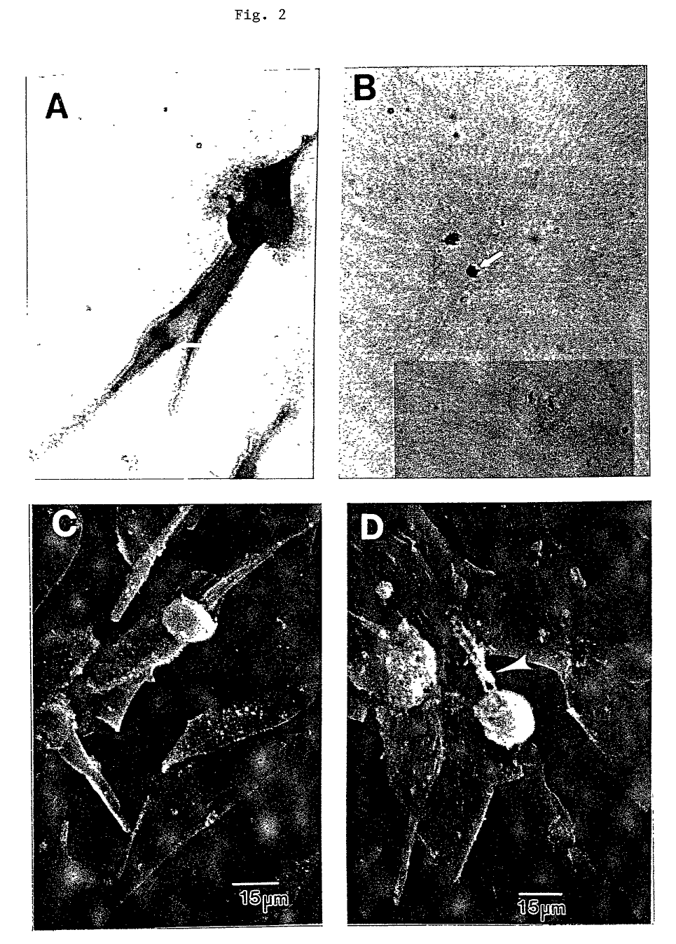

CHO cells and E. intestinalis were maintained as described above in Example 1. To analyze the developmental stages of E. intestinalis in CHO cells, the culture plates seeded with CHO cells were incubated for 48 h in medium containing 5% FCS until the monolayer reached over 55% confluency. The medium was removed and replaced with fresh medium containing 2% FCS. An inoculum containing 10⁵ spores was then added to each plate and the plates incubated as described above for 1, 2, 3, 4, 5 h, 5 day, and 10 day. At each time, one sample was fixed in phosphate buffered Trump's fixative for one h and dehydrated in 50% ethanol for 15 min, 75% ethanol for 15 min, 95% ethanol, twice for 15 min each, and absolute ethanol, twice for 30 min each. The samples was then critical point dried, sputter coated and examined. Most binding of E. intestinalis to host cells was observed within the first two hours, with maximum binding occurring at 2 h post inoculation. E. intestinalis spores used to infect CHO cells were not previously induced to protrude polar tubules, and as anticipated, free spores with a protruding tube were rarely seen. Fewer exospores were observed 3 h post infection and none at 4 h post infection, suggesting that infection was completed by 4 h post infection. It is noteworthy that at the same time period isolated vacuoles with gram positive spores were visible in samples processed as described above in Example 1. (See FIG. 2)

### EXAMPLE 3

Detection of Microsporidia in CHO Cells by Immunofluorescent Assay

A. Preparation of Rabbit Antiserum to E. intestinalis Exospores

Rabbit antiserum against E. intestinalis was prepared by immunization of two rabbits with E. intestinalis spore extract obtained by sonication of spores on ice, 4 times for 30 seconds with 30 seconds cooling in between. The rabbits were immunized subcutaneously with an emulsion of sonic extract (100 μl of a stock suspension containing 10⁵ spores/ml) mixed with an equal volume of Freund's complete adjuvant followed by three booster immunization with sonic extract mixed with an equal volume of Freund's incomplete adjuvant. Injections were given at three week intervals, and antiserum was collected after the fourth injection. Immunodot analysis of rabbit antiserum performed as described in Dao, M. L. 1985. An Improved Method of Antigen Detection on Nitrocellulose: In Situ Staining of Alkaline Phosphatase Conjugated Antibody. J. Immunol. Methods 82: 225-231.

Antiserum was shown to react with Escherichia coli extract and RK13 cell extract. After adsorption with E. coli antigens and uninfected RK13 cells that were immobilized onto a piece of nitrocellulose filter the antiserum only reacted against E. intestinalis spore extract and infected RK13 cells. Adsorbed rabbit antiserum was treated with 1:10 volume of chloroform for delipidation and sterilization, and stored in aliquots at −20° C. until use.

B. Inoculation and Detection

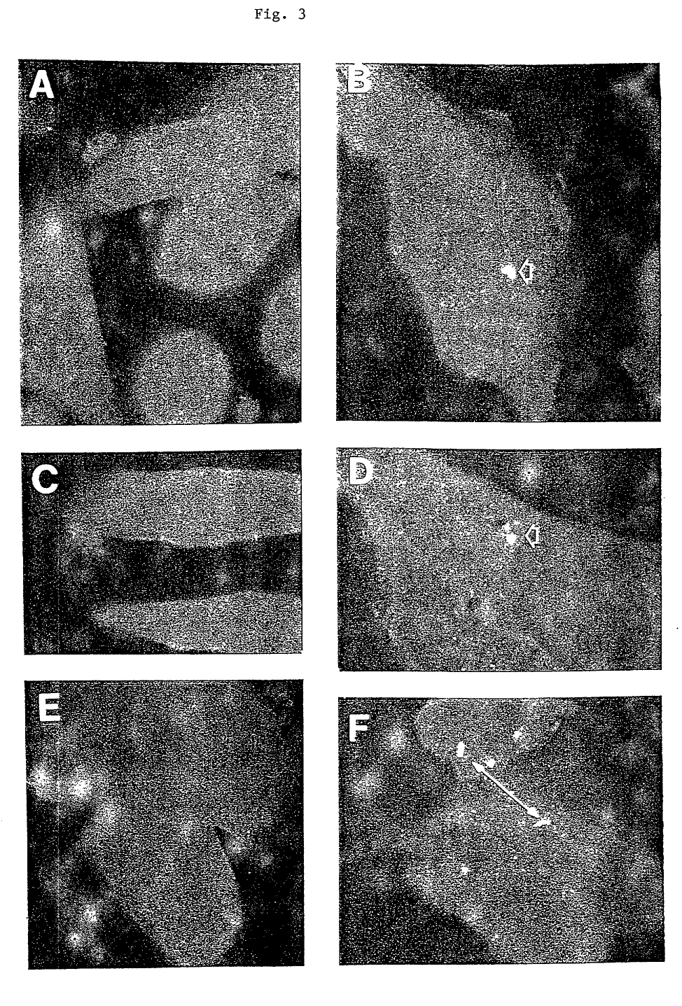

CHO cells were prepared, inoculated with spores obtained from CHO cells as described in Example 7 below, and incubated as described above in Example 1 above. At 1, 5, and 10 days post-inoculation, the samples were fixed and incubated with the primary antibody rabbit number 4 lot 2 (1:250) for 30 minutes. The coverslips were rinsed in PBS for 10 minutes and stained with secondary antibody, goat anti-rabbit immunoglobulins conjugated to FITC (1:80) for 30 minutes followed by a wash in PBS for 10 minutes. The coverslips were then counterstained with Evans blue, 2 drops in 25 ml PBS, for 15 minutes, mounted and examined on an epifluorescence mircroscope. As shown in FIG. 3, IFA was useful in detecting small infected foci and was not as useful in detecting large vacuoles within deteriorating cells. It is believed that this pattern is attributed either to the fact that the antibody is to a protein that is present on the mature form of the spore or to the inability of the antibody to penetrate intact parasitophorous vacuoles.

### EXAMPLE 5

Detection of Microsporidia by Phase Contrast Microscopy

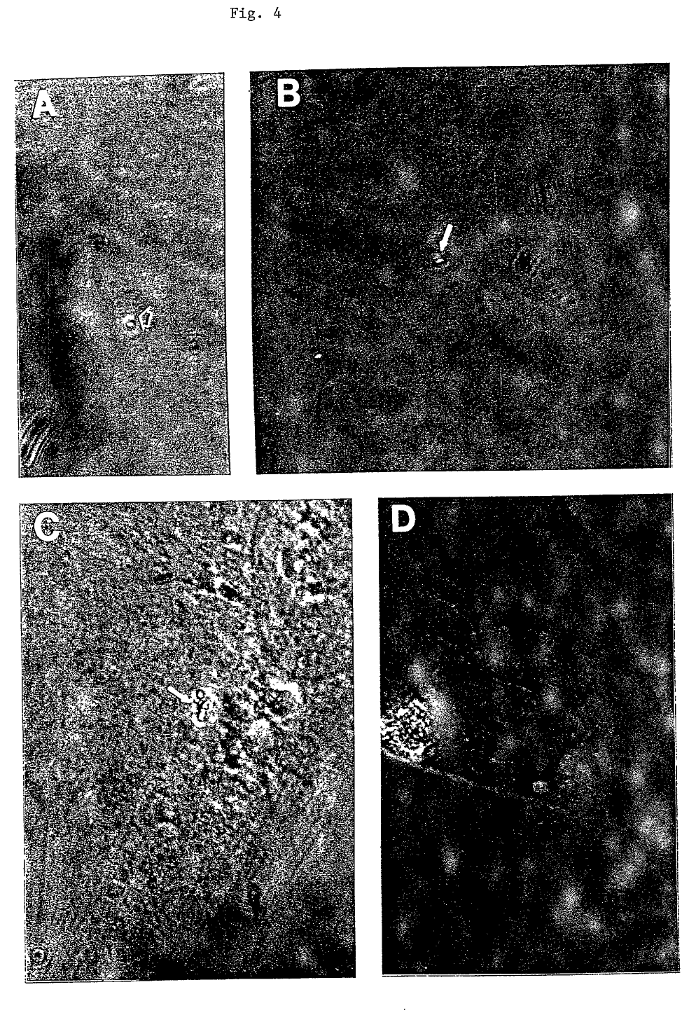

CHO cells were grown, inoculated with E. intestinalis and incubated as described above in Example 1. At 1, 3, 5, 7, and 9 days post-inoculation, the samples were fixed, inverted and sealed on microscope slides, and example with a phase contrast microscope. As shown in FIG. 4, infected vacuoles were detected within 24 hours after inoculation.

### EXAMPLE 6

Enhanced Infection of CHO Cells by E. intestinalis in Presence of Rabbit Antiserum to Exospore Extract

Using the same culture conditions as described above, various volumes (100, 200, 400, and 500 μl) of rabbit anti-E. intestinalis antiserum were added to separate cultures concomitantly with the E. intestinalis spores. After 24 h, the coverslips were stained with the Gram stain and observed as described above. The results showed massive infection of CHO cells Enhanced infection by antibody was in a dose-dependent manner, with the highest proportion of infected cells obtained with 400 μl and 500 μl of antiserum

TABLE 2
Antibody Influence on Infectivity

| CHO+ | 1 hour | 2 hours | 3 hours | 4 hours | 5 hours |
|---|---|---|---|---|---|
| Spores alone | − | − | − | + | + |
| Spores + Ab | + | + | + | + | ++ |

Table 2. Observation of foci in infected CHO cells at various intervals post infection. Ab = 300 μl of polyclonal antibody.

### EXAMPLE 7

Propagation of Microsporidia in CHO cells.

50 cm² flasks were seeded with 10⁶ CHO cells. At 48 hours after seeding, the flasks were inoculated with 10⁶ E. intestinalis spores and incubated as described above in Example 1. At 5, 7, 9, and 11 days after inoculation supernatant was collected from two flasks and the infected monolayers trypsinized. The supernatant was centrifuged for 5 minutes at 1000×g, the clarified supernatant decanted, and the pellet resuspended in 1 ml PBS. The suspended pellets were sonicated on ice twice for 30 seconds each. Following an additional 30 seconds on ice, the sonicate was diluted to 5 ml PBS and a 100 μl sample removed and the number of spores per sample determined with a hemocytometer. Spores isolated from infected CHO cells were identified by phase contrast microscopy, DAPI staining, and immunofluorescence assay.

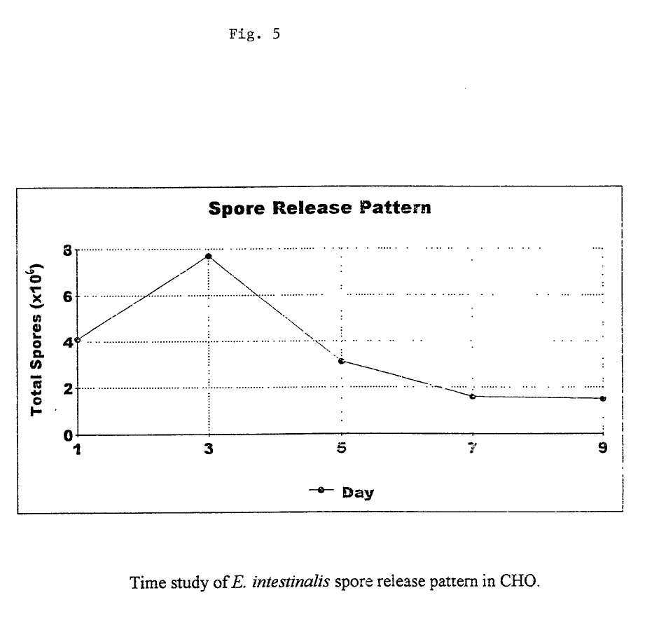

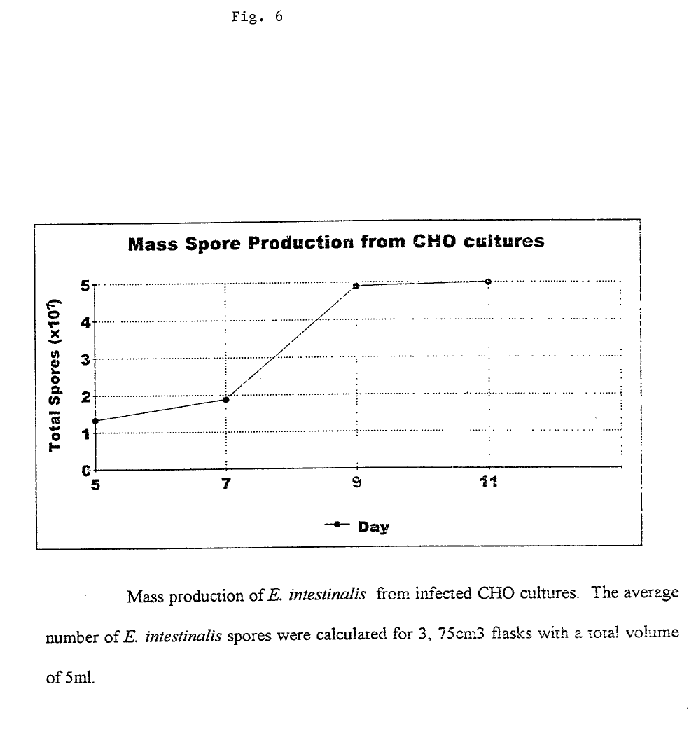

As shown in FIG. 5, the full life cycle of E. intestinalis is achieved by 72 hours post inoculation, at which time large numbers of spores are released into the medium. Currently only a few cell lines, including E6 and RK13 cells, have been shown capable of supporting E. intestinalis growth in vitro. The majority of these cell lines require long periods of incubation, typically from one to four months, to achieve massive infectivity and to obtain a continuous supply of spore. In contrast, with the present method massive quantities of spores were produced in only 11 days post infection. (See FIG. 6) In a two week period the number of spores produced by the present method is equivalent to those obtained in 4 month culture using different cell lines. Thus, the present method allows for a much more rapid production of spores than standard methods.

### EXAMPLE 8

Detection of Infectious Forms of C. parvum with DAPI Staining.

Purified oocysts (20-100 μl of 1×10⁸/ml) of C. parvum were incubated in fresh excystation fluid consisting of 0.5% trypsin, 1.5% sodium taurocholate and 1 % HBSS, pH 2.6, in 1×PBS for 20 minutes in a 37° C. waterbath with manual mixing every 4-6 min. After incubation, excysted oocysts were pelleted by centrifugation at 14,000 rpm for 4 min in a microcentrifuge. The pellet was resuspended in 1 ml RPMI-1640 culture medium containing 2% fetal calf serum.

Culture medium was aspirated from CHO cell monolayers grown to 60-70% and replaced with excysted oocyst media. This procedure, modified from Meloni and Thompson, confers three advantages over many other methods commonly used: (1)it is simple and requires little time (20 min.); (2) sporozoites are not exposed to stresses such as filtration and centrifugation associated with sporozoite purification; and (3) once the sporozoites excyst, they are in immediate contact with the CHO cells.

Cultures were then incubated in a 37° C., 5% CO₂ incubator for 90 min, after which additional medium was added and the monolayers were incubated for 48 h. After incubation, the culture medium was removed and the monolayers were washed with 1× PBS and fixed with cold 100% methanol for 10 min. at −20° C.

Using DAPI (4′,6-diamidino-2-phenylindole) to stain nucleic acids nonspecifically, we observed large, brightly-stained CHO cell nuclei,, surrounded by much smaller brightly-staining bodies within the cytoplasm, indicative of C. parvum intracellular stages. No infected foci could be seen in negative controls, consisting of uninfected CHO cells, and CHO cells inoculated with killed oocysts. Optimal detection was seen at 48 hrs. post-infection, however intracellular stages were also detectable within 24 hrs. Thus, the presence of infectious oocysts can be rapidly detected using the present method.

Quantification of Infection

Infection was carried out as described above using 2000-5000 oocysts per cm² of culture area and infected monolayers were examined by epifluorescence microscopy. Infection was quantified by 20 random oil field counts of infected foci located within the cytoplasm of CHO cells. Counts were initially done at 24, 48, and 60 hrs. after infection, with 48 hrs. showing the optimal results regarding number of foci per field. Counts analyzed per random field after 48 hrs. showed the following numbers: 1.2, 1.3, 1.25, 0.90, 0.85, 1.25, 2.4, 1.65, 1.9, and 1.5 foci per field. These numbers averaged 1.42±0.47 C parvum foci per field, 48 hrs. after infection.

### EXAMPLE 9

Detection of C. parvum by IFA Staining of Infected CHO Cells

Infected foci were detected in CHO cells by labeling intracellular developmental stages of the parasite with a primary antibody specific for a protein on C. parvum sporozoite membranes and a secondary fluorescein isothiocyantate-conjugated (FITC) antibody.

A. Polyclonal Antibody Production

Polyclonal antibody was prepared by hyperimmunizing rabbits subcutaneously with the purified C. parvum sporozoite membranes. Five microliters of homogenized concentrated sporozoite membrane (200 μg/n-A) in 45 μl 1×PBS (1:10 dilution) was administered subcutaneously into three rabbits. The initial injections consisted of 100 μl of Freund's complete adjuvant (Sigma Chemical Company, St. Louis, Mo.) with 100 μl of sporozoite membrane. Immunizations were continued every 2 weeks thereafter with Freund's incomplete adjuvant (Sigma) and sporozoite membrane as boosters. Rabbits were bled through the ear vein. Blood was allowed to clot overnight at 4° C. before the hyperimmune serum was collected.

Antibodies were purified by Protein A-sepharose column chromatography. The pH of the crude serum antibody preparation was adjusted to pH 8.0 by adding a 1:10 volume of 1.0 M Tris (pH 8.0). The antibody solution was passed through an Affi-Gel® Protein A column (Bio-Rad. Laboratories, Hercules, Calif.). The beads were washed with 10 column volumes of 100 mM Tris (pH 8.0). Beads were then washed with 10 column volumes of 10 mM Tris (pH 8.0). The column was eluted with 100 mM glycine (pH 3.0), adding approximately 500 μl per sample. The eluate was collected in 500 μl fractions into 1.5-ml microfuge tubes containing 50 μl of 1 M Tris, pH 8.0.

Antibodies were absorbed against E. coli of the JM109 strain, and against CHO cells. JM109 cells were grown up overnight in 15-ml conical tubes then transferred to 500 ml Luria Bertani (LB) broth and grown up overnight. Cells were centrifuged for 10 min at 10,000 g using a Sorvall RC-5B centrifuge with a GS-A rotor (Sorvall Inc. Newtown, Conn.). The pellet was resuspended in 5 ml 1×PBS, transferred to 50-ml centrifuge tubes, then sonicated on ice 60 sec. 3 times each using the Vibracell® Sonicator (Sonics &Materials, Danbury, Conn.), with an output of 60 W. Two milliliters of the sonicated extract was transferred to 2-ml microfuge tubes and centrifuged at 14,000 rpm using an Eppendorf 5415C microcentrifuge (Brinkman Instruments, Inc. Westbury, N.Y.). The wash was repeated in 1×PBS and the pellet was resuspended in 2 ml antiserum followed by incubation overnight at 4° C., in the presence of 1% sodium azide. The tube was centrifuged at 14,000 rpm for 3 minutes and the supernatant (absorbed antisera) was collected and diluted 1:5 in 1×PBS.

The procedure was repeated for CHO cells which were trypsinized and resuspended in 1 × PBS. CHO cells were homogenized on ice by aspiration 6 times through a 3-cc syringe with a 25-gauge needle. Antiserum was incubated with homogenized CHO cells overnight at 4° C. Antibodies were tested after absorption by immunodot analysis.

B. Staining of CHO cells Infected with C. parvum.

After fixing infected CHO cells, monolayers were blocked with 2% BSA in PBS-T for 30 min. while shaking. After blocking, monolayers were incubated with prepared primary polyclonal rabbit anti-Cryptosporidium antibody (1:250) in PBS-T for 1 h while shaking. Monolayers were washed with PBS-T twice, 15 minutes each. Secondary goat anti-rabbit antibody conjugated to fluorescein isothiocyanate (1:80) (Sigma) was then added to monolayers and incubated 30 min. while shaking. After 2-15 min. washes in PBS-T, monolayers were counterstained with Quantafluor Evan's Blue Counterstain (Kaffestad Inc. Chaska, Minn.) for 10 min. Uninfected CHO cells, as well as CHO cells inoculated with killed oocysts were also stained.

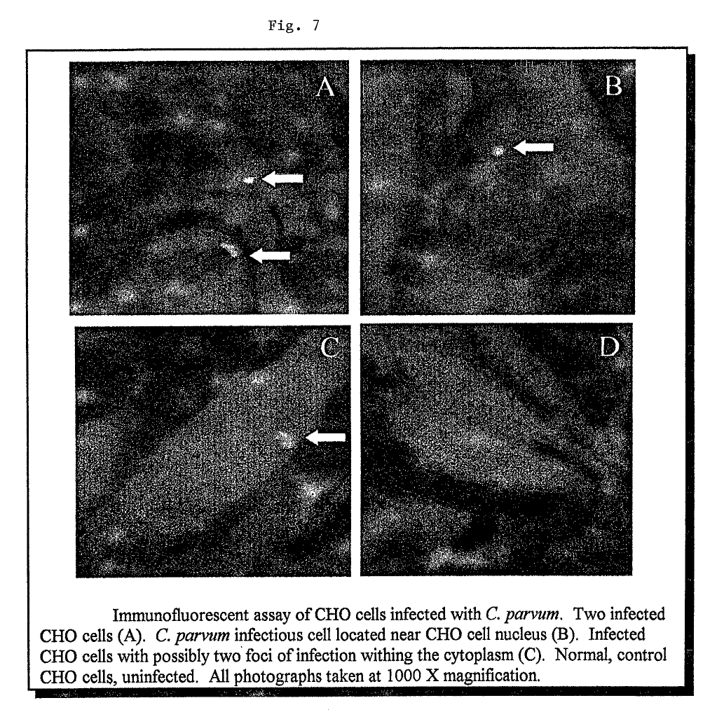

C. parvum infected foci fluoresced bright yellow-green against a background of CHO cells counterstained with Evan's blue, which stains the cells red. Some cells contained numerous infectious stages, which are oftentimes located near the cell nuclei. (See FIG. 7) Uninfected CHO cells, and cells inoculated with killed oocysts showed no fluorescent C parvum stages.

What is claimed is:

1. A method for detecting the presence of infectious forms of intracellular pathogenic protozoa in clinical or environmental samples comprising the steps of
a) isolating the protozoa present in the sample;
b) adding an inoculum of said isolated protozoa to the medium of a culture of CHO cells, and
c) assaying for the presence of the protozoa in the CHO cells.

2. The method of claim 1 wherein said protozoa are isolated from said sample by contacting said sample with an antibody that binds to an outer membrane protein of said protozoa.

3. The method of claim of claim 2 wherein said method is for detecting the presence of an infectious form of a protozoa belonging to the phylum Microspora, and wherein said protozoa is isolated by contacting said sample with an antibody which binds to an outer membrane of a spore of said protozoa.

4. The method of claim 2 wherein said method is for detecting the presence of an infectious form of a protozoa belonging to the genus Cryptosporidium, and wherein said protozoa is isolated by contacting said sample with an antibody which binds to an outer membrane of an oocyst of said protozoa.

5. The method of claim 1 wherein step (c) is accomplished by microscopically examining said cells for infected foci.

6. The method of claim 1 wherein step (c) is accomplished using a stain which stains said protozoa.

7. The method of claim 1 wherein step (c) is accomplished using an immunofluorescent assay.

8. The method of claim 1 further comprising the step of identifying the species of said protozoa using a polymerase chain reaction.

9. The method of claim 1 further comprising the step of identifying the species of said protozoa using an immunofluorescent assay.

10. A method for propagating an intracellular pathogenic microorganism selected from the group consisting of an intracellular protozoa and intracellular bacteria comprising the steps of:
a) providing a culture of CHO cells;
b) adding an inoculum of the microorganism to the medium of said culture of said CHO cells: and
c) incubating said CHO cells in the presence of said inoculum for a time sufficient to allow the micro-organism to infect said cells; and
d) incubating said CHO cells under conditions which permit said micro-organism to replicate within said CHO cells.

11. The method of claim 10 wherein said method is for propagating an intracellular pathogenic protozoa and wherein the inoculum comprises said intracellular pathogenic protozoa.

12. The method of claim 11 wherein said inoculum comprises an intracellular pathogenic protozoa belonging to the phylum Microspora.

13. The method of claim 12 wherein said inoculum comprises a protozoa belonging to the genus Encephalitozoon.

14. The method of claim 10 further comprising the step of obtaining the micro-organism from the cells and medium in said culture.

15. A kit for detecting the presence of infectious forms of an obligate intracellular protozoa in environmental sample or a clinical sample comprising:
a) an antibody that binds to a protein on a spore or an oocyst of said protozoa; and
b) an aliquot of CHO cells.

16. A method for testing a candidate pharmacological agent for an intracellular bacteria or an intracellular protozoa, comprising the steps of:
a) providing a culture of CHO cells;
b) adding the candidate agent to the medium of the CHO cell culture;
c) inoculating the medium of the CHO cell culture with an inoculum of an intracellular pathogenic bacteria or an intracellular pathogenic protozoa; and
d) assessing the effect of the candidate agent on infectivity or replication of the bacteria or protozoa within the CHO cell culture.

17. The method of claim 16 wherein the method is for testing a candidate anti-microsporidial agent and wherein said inoculum comprises microsporidial spores.

18. The method of claim 16 wherein the method is for testing a candidate anti-cryptosporidial agent and wherein said inoculum comprises cryptosporidial oocytes or sporozoites.
